(19) United States
(12) Patent Application Publication
(10) Pub. No.: US 2024/0189418 A1
(43) Pub. Date: Jun. 13, 2024

(54) VIRUS VACCINE BASED ON VIRUS SURFACE ENGINEERING PROVIDING INCREASED IMMUNITY

(71) Applicant: THE INDUSTRY & ACADEMIC COOPERATION IN CHUNGNAM NATIONAL UNIVERSITY (IAC), Daejeon (KR)

(72) Inventor: Hyun-Jin SHIN, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 18/553,277

(22) PCT Filed: Mar. 30, 2022
(86) PCT No.: PCT/KR2022/004491

§ 371 Date: Sep. 29, 2023

(30) Foreign Application Priority Data
Mar. 31, 2021 (KR) 10-2021-0042027
Oct. 26, 2021 (KR) 10-2021-0143999

(51) Int. Cl. A61K 39/215; A61K 39/00; A61P 31/14; C07K 14/005

(52) U.S. Cl. CPC A61K 39/215; A61P 31/14; C07K 14/005; A61K 2039/5252; A61K 2039/5254; A61K 2039/5258; C07K 2319/30; C12N 2770/10034; C12N 2770/24134

(57) ABSTRACT

The present disclosure relates to an immune-enhanced virus vaccine based on virus surface engineering. A linker peptide according to one aspect has the property of being attachable to a virus, and may be used as a linker that may effectively bind an immune-enhancing substance, which activates the immune system, to the surface of the virus, and thus may improve the immunogenicity of the vaccine. By incorporating the linker peptide into virus surface engineering technology, an immune-enhancing substance may be attached to the surface of the virus, which may be useful in an immune-enhanced vaccine platform.

### TECHNICAL FIELD

The present disclosure relates to an immune-enhanced virus vaccine based on virus surface engineering. This application claims priority to Korean Patent Application No. 10-2021-0042027, filed on Mar. 31, 2021, and Korean Patent Application No. 10-2021-0143999, filed on Oct. 26, 2021, the disclosures of which are hereby incorporated by reference.

### BACKGROUND ART

Due to the increase in overseas travel, the influx of endemic diseases prevalent in tropical and subtropical regions into the country is increasing, and in particular, imported contagious diseases such as dengue fever and novel coronavirus are causing hundreds of millions of patients worldwide every year. Not only in humans but also in animals, the economic damage is severe due to domestic diseases such as foot-and-mouth disease and African swine fever, as well as contagious diseases imported from abroad, adversely affecting the national economy. In order to prevent these viral diseases, vaccine development is of utmost importance. Since this requires a lot of time and money, the development of a more efficient and highly effective vaccine manufacturing platform is very important.

In general, there are various forms of vaccines depending on the manufacturing method: first-generation vaccines, such as attenuated vaccine and inactivated vaccine; second-generation vaccines, such as subunit vaccine and toxoid vaccine; and third-generation vaccines, such as DNA, RNA, and recombinant virus vaccines. The efficacy of these vaccines is very diverse, and the immune activation efficacy of each form is very different. For example, while subunit vaccines and virus vaccines have excellent safety, immune-enhancing substances or adjuvants are required to improve immunogenicity. An adjuvant is a substance that increases the immune response to an antigen by inducing temporary activation of the immune system, and many vaccine companies increase vaccine efficacy by adding an adjuvant when manufacturing a vaccine. However, a certain adjuvant may cause the vaccine's immune response to occur too severely, triggering side effects such as an allergic reaction. Therefore, there is a need for a new vaccine manufacturing technology that may maximize the efficacy of the vaccine while minimizing the side effects caused by the use of an adjuvant, and may also shorten the vaccine manufacturing process by being methodologically simple. This requires the establishment of a fundamentally new virus vaccine development platform that may solve the shortcomings of existing virus vaccine development.

Meanwhile, an epitope of a virus usually consists of a spike protein on the surface of the virus or a protrusion protruding from the outside, and if an antibody binds to the surface protein of the virus, it may prevent the virus from binding to the receptor of the host cell. The virus surface protein has the highest immunogenicity and neutralizing ability and is most important in viral infection. For example, in the case of coronaviruses, the viral structural proteins of spike protein (S), membrane protein (M), and envelope protein (E) protrude from the surface of the virus.

### DISCLOSURE

### Technical Problem

Under such background, the inventors of the present disclosure have completed the present disclosure by manufacturing a novel linker peptide that has a high affinity for other proteins and may be attached to the surface of a virus, constructing a new vaccine manufacturing platform that introduces immune-enhancing substances to the surface of a virus using virus surface engineering technology in combination with the linker peptide, and verifying the excellent antigenicity of a vaccine composition prepared using the vaccine platform.

One aspect provides a linker peptide including an amino acid sequence of SEQ ID NO: 1.

Another aspect provides a fusion protein including: a linker peptide consisting of the amino acid sequence of SEQ ID NO: 1; and an immune-enhancing substance connected to the C-terminus of the linker peptide.

Another aspect provides a vaccine composition including an infectious virus-derived antigen; and the fusion protein as an active ingredient.

Another aspect provides a method of preventing or treating an infectious disease, including the process of administering the vaccine composition to a subject, other than a human.

### Technical Solution

One aspect is to provide a linker peptide consisting of the amino acid sequence of SEQ ID NO: 1.

As used herein, the term “peptide” may refer to a linear molecule formed by combining amino acid residues to each other by peptide bonds. The peptides may be prepared according to chemical synthesis methods known in the art, in particular solid phase synthesis technique or liquid phase synthesis technique (U.S. Registered Pat. No. 5,516,891). As a result of diligent efforts to develop a peptide with biologically effective activity, the inventors of the present disclosure have identified a peptide consisting of the amino acid sequence of SEQ ID NO: 1. Here, biologically effective activity may refer to a connection or binding to the surface or at least any one region of a virus, while maintaining the intrinsic antigenicity of the virus.

As used herein, the term “linker” or “linker peptide” refers to a peptide that connects distinct polypeptide regions, desirably a peptide that may directly or indirectly connect a virus surface protein to an immune-enhancing substance.

The linker peptide may be consisted of a sequence of 20 to 30 amino acids, and may include the amino acid sequence represented by SEQ ID NO: 1, and desirably may be consisting of the amino acid sequence represented by SEQ ID NO: 1. In addition, the amino acid sequence may include not only the amino acid sequence consisting of SEQ ID NO: 1, but also, without limitation, an amino acid sequence showing 80% or more, specifically 90% or more, more specifically 95% or more, more specifically 98% or more, and most specifically 99% or more homology to the sequence, and which exhibits substantially the same or corresponding efficacy as the protein. In addition, it will be apparent to those skilled in the art that, as long as the amino acid sequence has such homology, amino acid sequences in which some sequences are deleted, modified, substituted, or added are included within the scope of the present disclosure.

As used herein, the term “homology” refers to the degree of similarity of the nucleic acid sequence encoding a protein or the amino acid sequence consisting the protein, such that if the homology is sufficiently high, the expression product of the gene and the protein may have the same or similar activity. the homology may be expressed as a percentage according to the degree of match to a given amino acid sequence or nucleic acid sequence, and may be calculated, for example, using standard software for calculating parameters such as score, identity, and similarity, etc., specifically BLAST 2.0, or by comparing the sequences by hybridization experiments under defined stringent conditions, wherein the appropriate hybridization conditions defined are within the scope of the art and may be determined by methods well known to those skilled in the art (for example, J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York).

The linker peptides have a high affinity for other proteins and, in particular, may be attached to the surface of a virus and thus may be used in the development of vaccine compositions with improved immunogenicity over typical virus vaccines by linking immune-enhancing substances to virus surface proteins. The linker peptides may be used, for example, to link or conjugate immune-enhancing substances to viruses, virus-derived subunits or antigens, and virus-like particles. More specifically, the linker peptide may provide binding capacity to a virus-derived antigen while simultaneously maintaining the antigenicity derived from the antigen itself, thereby may contribute to improved efficacy as a vaccine composition.

Another aspect is to provide a fusion protein including: a linker peptide consisting of the amino acid sequence of SEQ ID NO: 1; and an immune-enhancing substance connected to the C-terminus of the linker peptide.

The same parts of the description above applies equally to the fusion protein.

As used herein, the term “immune-enhancing substance” or “adjuvant” refers to a substance which may assist an immunogen in the formation of an immune response, which may act through several mechanisms, such as one or more mechanisms including increasing the biological or immunological half-life of an antigen; improving antigen delivery to an antigen-providing sequence; improving antigen processing and provision by an antigen-providing cell; and inducing the production of immunomodulatory cytokines.

The immune-enhancing substances are not particularly limited, but aluminum hydroxide, aluminum phosphate or other aluminum salt, calcium phosphate, DNA CpG motif, monophosphoryl lipid A, cholera toxin, Escherichia coli heat-inactivated toxin, pertussis toxin, muramyl dipeptide, Freund's incomplete adjuvant, MF59, SAF, Immunostimulatory complexes, liposomes, biodegradable microspheres, saponins, non-ionic block copolymers, muramyl peptide analogs, polyphosphazenes, synthetic polynucleotides, Fc regions of antibodies, flagellin, IFN-γ, interleukin-2 (IL-2) or interleukin-12 (IL-12), etc. may be used alone or in combination of two or more, and desirably, one or more selected from the group consisting of the Fc region of an antibody, flagellin, and interleukin-2 (IL-2) may be used.

The N-terminus of the linker peptide in the fusion protein may be for binding to a virus surface protein, and the C-terminus of the linker peptide may be for binding to an immune-enhancing substance. Desirably, the linker peptides may be arranged in the order of (virus surface protein)-(linker peptide)-(immune-enhancing substance), thereby may enhance the immunogenicity of the virus.

According to an embodiment, by using an antigenic peptide that may induce a vaccine response against various viruses such as PEDV, PRRSV, and SARS-COV-2, and an Fc-derived protein of IgG as an immune-enhancing substance to effectively induce the formation of antibodies to the antigenic peptide, the increased immunogenicity of a vaccine composition that has an immune-enhancing substance attached to a virus surface by a linker peptide according to one aspect was confirmed. Therefore, the linker peptide may be utilized in compositions and methods, etc. for preventing or treating infectious diseases caused by infection with a virus.

Another aspect is to provide a polynucleotide encoding the fusion protein, or a recombinant vector including the polynucleotide.

As used herein, the term “polynucleotide” refers to a polymeric substance to which nucleotides are conjugated, such as DNA, which encodes genetic information.

In the present disclosure, a nucleic acid sequence consisting a polynucleotide encoding a linker peptide includes, without limitation, not only a nucleic acid sequence encoding the amino acid set forth in SEQ ID NO: 1, but also a nucleic acid sequence showing 80% or more, specifically 90% or more, more specifically 95% or more, more specifically 98% or more, and most specifically 99% or more homology to the sequence, as well as a nucleic acid sequence constituting a polynucleotide encoding a protein exhibiting substantially the same or corresponding potency as each of the proteins.

In addition, the polynucleotide encoding the linker peptide may be subject to various modifications in the coding region without changing the amino acid sequence of the protein expressed from the coding region, taking into account the preferred codon in the organism in which the protein is to be expressed due to degeneracy of the codon. Therefore, the polynucleotides may be included without limitation in the nucleic acid sequence encoding the respective proteins. In addition, a probe which may be prepared from the disclosed sequences, for example, a sequence that hybridizes under stringent conditions to a complementary sequence for all or part of the polynucleotide sequence, and encodes a protein that was the same activity as the above protein may be included without limitation.

The term “stringent condition” refers to a condition that enable specific hybridization between polynucleotides. These conditions are described in detail in the literature (for example, J. Sambrook et al., same as above). For example, hybridization between genes with high homology, 40% or more, specifically 90% or more, more specifically 95% or more, more specifically 97% or more, and especially specifically 99% or more homology, and conditions in which genes with less homology do not hybridize, or washing conditions for a typical Southern hybridization such as a salt concentration and temperature equivalent to 60° C. 1×SSC, 0.1% SDS, specifically 60° C. 0.1×SSC, 0.1% SDS, more specifically 68° C., 0.1×SSC, 0.1% SDS, which are the conditions for washing once, specifically 2 to 3 times, may be listed.

Hybridization requires that the two polynucleotides have complementary sequences, although mismatch between bases are possible depending on the degree of stringency of the hybridization. The term “complementary” is used to describe the relationship between nucleotide bases that may hybridize with each other. For example, with respect to DNA, adenosine is complementary to thymine and cytosine is complementary to guanine. Therefore, in addition the present disclosure may include substantially similar polynucleotide sequences as well as isolated polynucleotide fragments that are complementary to the entire sequence.

Specifically, a polynucleotide that has homology may be detected using hybridization conditions including a hybridization process at a Tm value of 55° C. and using the conditions described above. In addition, the Tm value may be 60° C., 63° C., or 65° C., but is not limited thereto and may be appropriately adjusted by a person skilled in the art depending on the purpose. The appropriate degree of stringency to hybridize a polynucleotide depends on the length of the polynucleotide and the degree of complementarity, variables that are well known in the art.

As used herein, the term “vector” refers to a vector that may express a target protein in a suitable host cell, and a genetic construct including regulatory elements operably connected to cause expression of the gene insert. A vector according to an embodiment may include expression regulatory elements such as a promoter, an operator, an initiation codon, a termination codon, a polyadenylation signal, and/or an enhancer, and the promoter of the vector may be constitutive or inducible. In addition, the vector may be an expression vector, which may stably express the fusion protein in a host cell. The expression vector may be any typical one used in the art for expressing a foreign protein in a plant, animal or microorganism. The recombinant vector may be constructed by a variety of methods known in the art. For example, the vector may include a selectable marker for selecting host cells containing the vector, and if the vector is replicable, may include an origin of replication.

The vector include a promoter operable in an animal cell, for example, a mammalian cell. According to an embodiment, suitable promoters include promoters derived from mammalian viruses and promoters derived from the genome of mammalian cells, for example may include Cytomegalovirus (CMV) promoter, U6 promoter and H1 promoter, Murine Leukemia Virus (MLV) long terminal repeat (LTR) promoter, adenovirus early promoter, adenovirus late promoter, vaccinia virus 7.5K promoter, SV40 promoter, tk promoter of HSV, RSV promoter, EF1 alpha promoter, metallothionein promoter, beta-actin promoter, promoter of human IL-2 gene, promoter of human IFN gene, promoter of human IL-4 gene, a promoter of a human lymphotoxin gene, a promoter of a human GM-CSF gene, a human phosphoglycerate kinase (PGK) promoter, a mouse phosphoglycerate kinase (PGK) promoter, and a survivin promoter.

In addition, in the vector, the polynucleotide sequence encoding the above-described fusion protein may be operably connected to a promoter. As used herein, the term “operably connected” refers to a functional conjugation between a nucleic acid expression regulatory sequence (for example: a promoter, signal sequence, or array of transcriptional regulator binding sites) and another nucleic acid sequence, whereby the regulatory sequence regulates the transcription and/or translation of the other nucleic acid sequence.

Another aspect is to provide a host cell transformed with the recombinant vector.

As used herein, the term “transformation” refers to a molecular biological technique in which a fragment of DNA chain or a plasmid carrying a foreign gene of a different kind from that carried by the original cell is introduced into a cell and changes the genotype of the cell by binding to the DNA present in the original cell. For the purposes of the present disclosure, transformation refers to a polynucleotide encoding a fusion protein including an amino acid sequence consisting of SEQ ID NO: 1 and an immune-enhancing substance connected to the C-terminus of the amino acid sequence is inserted into a host cell and produced.

The host cell may desirably be any one selected from the group consisting of, but not limited to, microorganisms such as bacteria (E. coli) or yeast, etc., CHO cells, F2N cells, and HEK293 cells.

Another aspect is to provide a vaccine composition including an infectious virus-derived antigen; and a fusion protein including a linker peptide consisting of the amino acid sequence of SEQ ID NO: 1 and an immune-enhancing substance connected to the C-terminus of the linker peptide.

The same parts of the description above applies equally to the vaccine composition.

As used herein, the term “vaccine” refers to a biological agent containing an antigen that provides immunity to a living body, and an immunogen or antigenic substance that creates immunity in a living body by injection or oral administration to a human or animal to prevent an infectious disease. In vivo immunity is largely divided into autoimmunity, which is immunity obtained automatically in the body after infection with a pathogen, and passive immunity, which is obtained by an externally injected vaccine. While autoimmunity is characterized by a long period of production of antibodies related to immunity and sustained immunity, passive immunity through vaccines acts immediately in treating infectious diseases, but has the disadvantage of being less durable. The term “vaccine” may be used interchangeably with the term “immunogenic composition” and may be, for example, an immunogenic composition against an infection of porcine epidemic diarrheal disease virus, or porcine genital and respiratory syndrome virus, but is not limited thereto.

As used herein, the term “immunogen” or “antigenic substance” may be any one selected from the group consisting of a peptide derived from the virus, a polypeptide, a lactic acid bacteria expressing the polypeptide, a protein, and a lactic acid bacteria expressing the protein, an oligonucleotide, a polynucleotide, and a recombinant virus. As a specific example, the antigenic substance may be in the form of an inactivated whole or partial virus preparation, or in the form of an antigen molecule obtained by typical protein purification, genetic engineering techniques, or chemical synthesis.

In an embodiment, the virus is not particularly limited, but may be an RNA-type virus or a DNA-type virus.

The RNA virus is a general term for a virus that has RNA as a gene, and include (+) chain RNA, complementary (−) chain RNA, and double-stranded RNA as genes in a viral particle, virus with only one molecule of RNA (corona virus, paramyxovirus), virus with two RNA molecules of the same type (retrovirus), and virus with eight different RNA molecules as genes (influenza virus), etc. Usually, RNA polymerase (DNA polymerase in the case of retroviruses) exists using RNA as a template.

A DNA virus is classified as either a circular DNA virus or a linear DNA virus, depending on the shape of their gene. Linear DNA viruses include parvovirus, which has single-stranded linear DNA in its viral particles, and adenovirus, herpesvirus, and poxvirus, etc., which have double-stranded chain linear DNA. Most have special repetitive sequences at the ends of their genomes, and each exhibits a unique pattern of infection and proliferation due to differences in genome structure or size. A DNA virus, which has a circular DNA molecule as genomes, are broadly classified into two virus families. That is, the papovirus family virus (polyomavirus, SV40, papillomavirus, etc.), which has a double-helical closed loop DNA molecule as its genome, and the Hepadnavi-ridae virus family virus (hepatitis B virus, Woodchuck hepatitis virus, etc.), which has a double-helical loop DNA molecule including a single-stranded portion as its genome.

As used herein, the virus to which the linker peptide may bind to the surface protein is not particularly limited, and may include all RNA-type viruses or DNA-type viruses as described above.

In an embodiment, the infectious virus-derived antigen may be an antigen derived from Porcine epidemic diarrhea virus, Porcine reproductive and respiratory syndrome virus, Dengue virus, Japanese encephalitis virus, Zika virus, Ebola virus, Rotavirus, West Nile virus, Yellow fever virus, Adenovirus, BK virus, Smallpox virus, Severe fever with thrombocytopenia syndrome virus, Herpes simplex virus, Epstein-Barr virus, Hepatitis A virus, Hepatitis B virus, Hepatitis C virus, Hepatitis D virus, Hepatitis E virus, Hantan virus, or Cytomegalovirus, but is not limited thereto.

In an embodiment, the vaccine composition may be a live attenuated vaccine, an inactivated vaccine, a subunit vaccine, or a virus like particle vaccine.

As used herein, the term “Virus-Like Particle (VLP)” may refer to a non-infectious viral subunit that may or may not contain a viral protein. For example, a virus-like particle may refer to a recombinant protein with a virus-like conformation, wherein the virus-like particle self-assembles into a virus-like conformation by binding to structural proteins of the virus, but the genes of the virus may not be incorporated into the virus-like particle during the assembly procedure. Virus-like particle with the above property has a form very similar to an actual virus, thus may exhibit high immunogenicity when injected into the body, and since the virus-like particle does not include a viral gene, and the virus-like particle may act as a safe antigen that cannot proliferate in the body.

The virus-like particle may include a spike protein, membrane protein, envelope protein, and nucleocapsid protein of a virus, for example, a corona virus. Here, the spike protein is a structural protein present on the surface of the virus and consists of club-shaped protrusions. The protein is known to bind to glycoprotein receptors on host cells, which may cause fusion of the cell membrane and the viral outer membrane and the production of neutralizing antibodies. In addition, the nucleocapsid protein is known to be present in the interior of the envelope and are involved in the cellular immune response.

The vaccine composition may additionally include a pharmaceutically acceptable excipient, diluent, or carrier. The term “pharmaceutically acceptable excipient, diluent or carrier” may refer to an excipient, diluent or carrier that does not irritate living organisms and does not inhibit the biological activity and properties of the injected compound. Here, “pharmaceutically acceptable” means that it does not inhibit the activity of the active ingredient and does not have any toxicity beyond what the subject of application (prescription) may adapt to.

Suitable carrier for a vaccine is known to those skilled in the art and include, but are not limited to, protein, sugar, etc. The carrier may be an aqueous solution or non-aqueous solution, suspension or emulsion. Structured or unstructured organic or inorganic polymer, etc. may be used as an immune adjuvant to increase immunogenicity. Immune adjuvants are generally known to play a role in promoting immune responses through chemical and physical binding to an antigen. As an immune adjuvant, an amorphous aluminum gel, oil emulsion, double oil emulsion, immunosol, etc. may be used. In addition, various plant-derived saponin, levamisole, CpG dinucleotide, RNA, DNA, LPS, and various types of cytokines, etc. may be used to promote the immune response. The immune composition as described above may be used as a composition for inducing an optimal immune response by combining various adjuvants and additives to promote immune response. In addition, compositions that may be added to the vaccine include a stabilizer, inactivator, antibiotic, preservative, etc. Depending on the route of administration of the vaccine, the vaccine antigen may also be mixed with distilled water, buffer solution, etc.

The vaccine compositions may be formulated and used in the form of an oral dosage form such as a pill, granule, tablet, capsule, suspension, emulsion, syrup, aerosol, etc., topical preparation, suppository, unit dosage ampoule, or injectable formulation in the form of multiple dosages each according to typical methods. The vaccine composition may be formulated with the addition of a diluent or excipient such as commonly used filler, extender, binder, lubricant, disintegrating agent, or surfactant, etc.

If the vaccine composition is prepared as a parenteral formulation, the vaccine composition may be formulated in the form of an injectable formulation, transdermal administration, nasal inhalant, and suppository along with a suitable carrier according to methods known in the art. When formulated as an injectable formulation, suitable carriers may include sterile water, ethanol, polyols such as glycerol or propylene glycol, or mixtures thereof, desirably Ringer's solution, phosphate buffered saline (PBS) containing triethanolamine, sterile water for injection, and isotonic solutions such as 5% dextrose, etc. may be used. When formulated for transdermal administration, the vaccine composition may be formulated in the form of an ointment, cream, lotion, gel, topical solution, paste, liniment, and aerosol, etc. For a nasal inhalant, the vaccine composition may be formulated in the form of an aerosol spray using a suitable propellant such as dichlorofluoromethane, trichlorofluoromethane, dichlorotetrafluoroethane, carbon dioxide, etc., when formulated as a suppository, the base used may be witepsol, tween 61, polyethylene glycol, cacao paper, laurin paper, polyoxyethylene sorbitan fatty acid esters, polyoxyethylene stearate, sorbitan fatty acid esters, etc.

The administration route of the vaccine composition may be via any general route as long as the vaccine composition may reach the target tissue, and more specifically, the vaccine composition may be selected from the group consisting of compositions for intramuscular administration, subcutaneous administration, intraperitoneal administration, intravenous administration, oral administration, dermal administration, ocular administration, and intracerebral administration.

The vaccine composition may be administered in a pharmaceutically effective amount, wherein the term “pharmaceutically effective amount” refers to an amount sufficient to treat or prevent a disease with a reasonable benefit/risk ratio applicable to medical treatment or prevention, and an effective dose level may be determined according to factors including severity of the disease, activity of a drug, age, weight, health, and sex of a patient, sensitivity of the patient to a drug, time of administration, route of administration, and elimination rate of the composition of the disclosure used, duration of treatment, the drugs used in combination or concurrently with the composition of the disclosure, and other factors well known in the medical field. The vaccine composition may be administered alone or in combination with ingredients known to exhibit preventive or therapeutic effects against known infectious diseases. It is important to consider all of the above factors and administer the amount that may achieve the maximum effect with the minimum amount without side effects.

The dosage of the vaccine composition may be determined by a person skilled in the art considering the purpose of use, the degree of addiction of the disease, the patient's age, weight, gender, pre-existing condition, or the type of substance used as an active ingredient, etc. For example, the vaccine compositions of the present disclosure may be administered at a dose of from about 0.1 ng to about 1,000 mg/kg per adult, desirably from 1 ng to about 100 mg/kg, and the frequency of administration of the compositions of the present disclosure is not specifically limited, but may be administered once daily or administered multiple times in divided doses. The dosage or frequency of administration is not intended to limit the scope of the present disclosure in any way.

Another aspect provides a method of preventing or treating an infectious disease, including the process of administering the vaccine composition to a subject. The same aspects as described above apply equally to the method.

As used herein, the term “prevent” refers to any act of inhibiting or delaying infection of an infectious disease and the development of the infectious disease by administration of the vaccine composition.

As used herein, the term “treatment” refers to any act by which the administration of a vaccine composition ameliorates or benefits a condition already caused by infection with an infectious disease.

As used herein, the term “infectious disease” refers to a viral infectious disease, desirably a disease caused by infection with a virus, but is not limited thereto.

A subject may include, without limitation, mammals, including a bovine, equine, sheep, pig, goat, camel, antelope, dog, cat, rat, livestock, human, etc., or farmed fish, etc. that has or at risk of developing a viral infection and a disease caused by the infection.

As used herein, the term “administration” refers to introducing a certain substance into a subject by an appropriate method, and the administration route of the vaccine composition of the present disclosure may be administered through any general route as long as the substance may reach the target tissue. Routes of administration may include, but not limited to, intraperitoneal administration, intravenous administration, intramuscular administration, subcutaneous administration, intradermal administration, oral administration, topical administration, intranasal administration, intrapulmonary administration, and rectal administration. However, since proteins are digested when administered orally, it is desirable for oral compositions to be coated with the active agent or formulated to protect them from decomposition in the stomach. In addition, a pharmaceutical composition may be administered by any device that may transport the active substance to target cells.

The vaccine composition may be administered as an individual therapeutic agent or administered in combination with other therapeutic agents and may be administered sequentially or simultaneously with typical therapeutic agents. And may be administered in single or multiple doses. Taking all of the above factors into consideration, it is important to administer an amount that may achieve maximum effect with the minimum amount without side effects, and may be readily determined by those skilled in the art.

### Advantageous Effects

According to one aspect, the linker peptide has a property of being able to be attached to a virus, which may be used as a linker that may effectively bind an immune-enhancing substance that activates the immune system to the surface of the virus, thereby may improve the immunogenicity of the vaccine. By incorporating the linker peptide into virus surface engineering technology, an immune-enhancing substance may be attached to the surface of the virus, which may make the linker peptide useful as an immune-enhanced vaccine platform.

### DESCRIPTION OF DRAWINGS

FIG. 1 shows the result of Western blotting confirming the expression of a linker peptide (VSE peptide) according to one aspect.

FIG. 2 confirms the adhesion activity of a linker peptide according to one aspect to the surface of the virus, wherein A of FIG. 2 schematically illustrates the formation and detection of antigen-antibody complexes to evaluate the adhesion activity to the virus surface, and B of FIG. 2 is a diagram quantitatively illustrating the results of evaluating the adhesion activity to the virus surface.

FIG. 3 shows the result of Western blotting confirming the expression of recombinant proteins (VSE-hFc, VSE-sFc) according to one aspect.

FIG. 4 confirms the adhesion activity of the recombinant protein according to one aspect to the surface of the virus, wherein A of FIG. 4 schematically illustrates the formation and detection of antigen-antibody complexes to evaluate the adhesion activity to the virus surface, and B of FIG. 4 is a diagram quantitatively illustrating the results of evaluating the adhesion activity to the virus surface.

FIG. 5 is a schematic illustration of a procedure for preparing a recombinant antigen (PEDV-VSE-sFc) according to one aspect.

FIG. 6 shows the result of confirming the level of IgG specific for PEDV present in the serum of a mouse after intraperitoneal administration of PEDV-VSE-sFc according to one aspect to the mouse.

FIG. 7 shows the result of confirming the level of neutralizing antibodies present in the serum of a mouse after intraperitoneal administration of PEDV-VSE-sFc according to one aspect to the mouse.

FIG. 8 is a diagram schematically illustrating the procedure of producing a recombinant antigen (DENV-VSE-hFc) according to one aspect.

FIG. 9 shows the results of confirming the level of IgG specific for DENV present in the serum of a mouse after intraperitoneal administration of DENV-VSE-hFc according to one aspect to the mouse.

FIG. 10 is a result of confirming the level of neutralizing antibodies present in the serum of a mouse after intraperitoneal administration of DENV-VSE-hFc according to one aspect to the mouse.

FIG. 11 shows the result of Western blotting confirming the expression of a recombinant antigen (PEDV-Fc) according to one aspect.

FIG. 12 shows the result of confirming the level of IgG present in the serum and colostrum of an animal model after administration of PEDV-Fc according to one aspect to the animal model.

FIG. 13 shows the result of confirming the level of neutralizing antibodies present in the serum of an animal model after administration of PEDV-Fc according to one aspect to the animal model.

FIG. 14 shows the result of Western blotting confirming the expression of a recombinant antigen (PRRSV-Fc) according to one aspect.

FIG. 15 shows the results of confirming the level of IgG present in the serum and colostrum of an animal model after administration of PRRSV-Fc according to one aspect to the animal model.

### BEST MODE

The present disclosure will be explained in more detail in the following embodiments. However, these embodiments are for illustrative purposes only and the scope of the disclosure is not limited to these embodiments.

### Example 1: Linker Peptide Production

A linker peptide (VSE peptide) with effective binding ability to the virus surface or virus-derived antigen was derived and prepared. The amino acid sequences of the linker peptides and the polynucleotide sequences encoding them are shown in Table 1 below.

TABLE 1

| NAME | SEQUENCE (5′→3′) | SEQ ID NO: |
|---|---|---|
| VSE peptide | TQEVYDTHDCATNGTIRPFKVLS | 1 |
| VSE polynucleotide | ACCCAAGAGGTGTACGACACCCACGACTGCGCCACC | 2 |
| | AACGGCACCATCAGACCTTTCAAGGTGCTGAGC | |

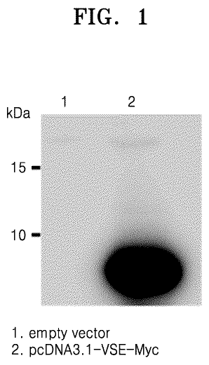

In this embodiment, the VSE polynucleotide was cloned into the pcDNA3.1-Myc-His vector, an expression vector for eukaryotic cells, and then expressed in CHO cells, and purified using Myc tag to obtain a VSE peptide as shown in FIG. 1.

### Example 2: Confirmation of Adhesion Activity to Virus Surface

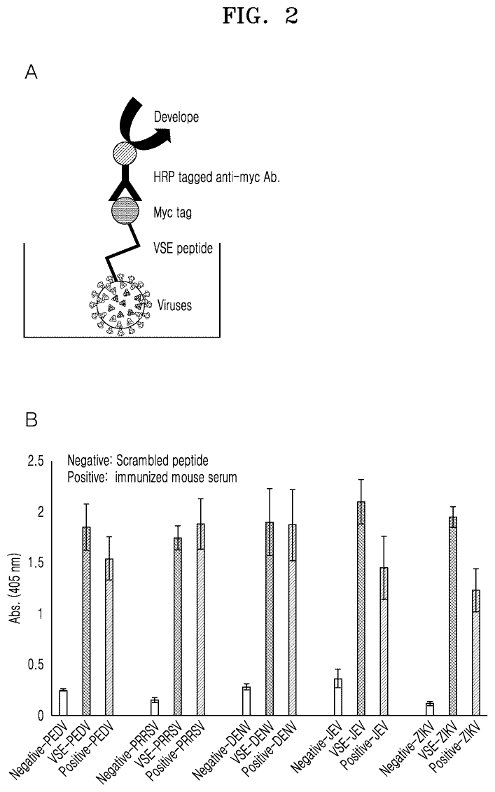

In this embodiment, the adhesion activity of the VSE peptide to the virus surface was confirmed by ELISA. Specifically, porcine epidemic diarrhea virus (PEDV), porcine reproductive and respiratory syndrome virus (PRRSV), dengue virus (DENV), Japanese encephalitis virus (JEV), or Zika virus (ZIKV) were each coated on the surface of an immunoplate, and the Myc-labeled VSE peptide (VSE-Myc tag) of Example 1 was added to induce an adhesion/binding reaction. Horseradish peroxidase (HRP)-labeled anti-Myc tag antibody was then added to induce the reaction, and the level of HRP was quantitatively detected to assess the adhesion activity to the virus surface (A of FIG. 2). Meanwhile, the negative control group was set as the group to react with scrambled peptide, and the positive control group was set as the group to use mouse serum immunized with each virus.

As a result, as shown in B of FIG. 2, the negative control group had a very low detection level of labeled HRP, while the group using the VSE peptide according to one aspect detected a level similar to that of the positive control group. These experimental results show that the VSE peptide according to one aspect has effective adhesion activity to the surface of the virus.

### Example 3: Confirmation of Adhesion Activity of Immune-Enhancing Substance to Virus Surface

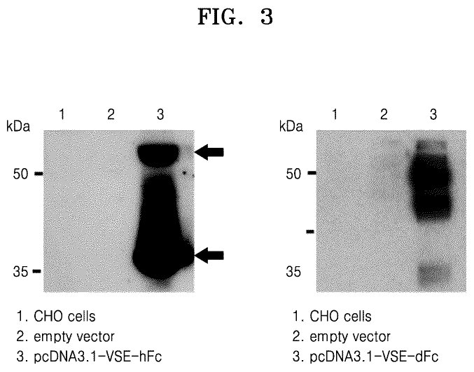

In this embodiment, the adhesion activity of the VSE peptide to the virus surface was utilized to attach or bind an immune-enhancing substance to the virus surface. Specifically, the VSE peptide and human Fc (VSE-hFc) or swine Fc (VSE-sFc) were cloned into a eukaryotic cell expression vector, pcDNA3.1-Myc-His vector, and expressed in CHO cells to obtain a recombinant protein, VSE-hFc or VSE-sFc, including an immune-enhancing substance, as shown in FIG. 3. The amino acid sequences of the recombinant proteins (VSE-hFc, VSE-sFc) and the polynucleotide sequences encoding them are shown in Table 2 and Table 3 below.

TABLE 2

| NAME | SEQUENCE (5′→3′) | SEQ ID NO: |
|---|---|---|
| VSE-human Fc | TQEVYDTHDCATNGTIRPFKVLSDKTHTCPPCPAPELLGGPSVFLFP | 3 |
| | PKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTIS | |
| | KAKGQPREPOVYTLPPSRDELTKNQVSLTCLVKGFYPSDIAVEWES | |
| | NGQPENNYKTTPPVLDSDGSFFLYSKLTVDKSRWQQGNVFSCSVM | |
| | HEALHNHYTQKSLSLSPGK | |
| VSE-human FC | ACCCAAGAGGTGTACGACACCCACGACTGCGCCACCAACGGCA | 4 |
| | CCATCAGACCTTTCAAGGTGCTGAGCGACAAAACTCACACATGC | |
| | CCACCGTGCCCAGCACCTGAACTCCTGGGGGGACCGTCAGTCTT | |
| | CCTCTTCCCCCCAAAACCCAAGGACACCCTCATGATCTCCCGGAC | |
| | CCCTGAGGTCACATGCGTGGTGGTGGACGTGAGCCACGAAGACC | |
| | CTGAGGTCAAGTTCAACTGGTACGTGGACGGCGTGGAGGTGCAT | |
| | AATGCCAAGACAAAGCCGCGGGAGGAGCAGTACAACAGCACGTA | |
| | CCGTGTGGTCAGCGTCCTCACCGTCCTGCACCAGGACTGGCTGA | |
| | ATGGCAAGGAGTACAAGTGCAAGGTCTCCAACAAAGCCCTCCCA | |
| | GCCCCCATCGAGAAAACCATCTCCAAAGCCAAAGGGCAGCCCCG | |
| | AGAACCACAGGTGTACACCCTGCCCCCATCCCGGGATGAGCTGA | |
| | CCAAGAACCAGGTCAGCCTGACCTGCCTGGTCAAAGGCTTCTATC | |
| | CCAGCGACATCGCCGTGGAGTGGGAGAGCAATGGGCAGCCGGA | |
| | GAACAACTACAAGACCACGCCTCCCGTGCTGGACTCCGACGGCT | |
| | CCTTCTTCCTCTACAGCAAGCTCACCGTGGACAAGAGCAGGTGG | |
| | CAGCAGGGGAACGTCTTCTCATGCTCCGTGATGCATGAGGCTCT | |
| | GCACAACCACTACACGCAGAAGAGCCTCTCCCTGTCTCCGGGTA | |
| | AA | |

TABLE 3

| NAME | SEQUENCE (5′→3′) | SEQ ID NO: |
|---|---|---|
| VSE-swine Fc | TQEVYDTHDCATNGTIRPFKVLSAYNTAPSVYPLAPCGRDVSDHNV | 5 |
| | ALGCLVSSYFPEPVTVTWNSGALSRVVHTFPSVLQPSGLYSLSSMVI | |
| | VAASSLSTLSYTCNVYHPATNTKVDKRVDIEPPTPICPEICSCPAAEV | |
| | LGAPSVFLFPPKPKLILMISRTPKVTCVVVDVSQEEAEVQFSWYVDG | |
| | VQLYTAQTRPMEEQFNSTYRVVSVLPIQHQDWLKGKEFKCKVNNKD | |
| | LLSPITRTISKATGPSRVPQVYTLPPAWEELSKSKVSITCLVTGFYPPD | |
| | IDVEWQSNGQQEPEGNYRTTPPQQDVDGTYFLYSKLAVDKVRWQR | |
| | GDLFQCAVMHEALHNHYTQKSISKTQGK | |
| VSE-swine Fc | ACCCAAGAGGTGTACGACACCCACGACTGCGCCACCAACGGCA | 6 |
| | CCATCAGACCTTTCAAGGTGCTGAGCGCCTACAACACAGCTCCA | |
| | TCGGTCTACCCTCTGGCCCCCTGTGGCAGGGACGTGTCTGATCA | |
| | TAACGTGGCCTTGGGCTGCCTTGTCTCAAGCTACTTCCCCGAGCC | |
| | AGTGACCGTGACCTGGAACTCGGGTGCCGTGTCCAGAGTCGTGC | |
| | ATACCTTCCCATCCGTCCTGCAGCCGTCAGGGCTCTACTCCCTCA | |
| | GCAGCATGGTGATCGTGGGGGCCAGCAGCCTGTCCACCCTGAGC | |
| | TACACGTGCAACGTCTACCACCCGGCCACCAACACCAAGGTGGA | |
| | CAAGCGTGTTGACATCGAACCCCCCACACCCATGTGTCCCGAAAT | |
| | TTGCTCATGCCCAGCTGCAGAGGTCCTGGGAGCACCGTCGGTCT | |
| | TCCTCTTCCCTCCAAAACCCAAGGACATCCTCATGATCTCCCGGA | |
| | CACCCAAGGTCACGTGCGTGGTGGTGGACGTGAGCCAGGAGGA | |
| | GGCTGAAGTCCAGTTCTCCTGGTACGTGGAGGGGGTACAGTTGT | |
| | ACACGGCCCAGACGAGGCCAATGGAGGAGCAGTTCAACAGCACC | |
| | TACCGCGTGGTCAGCGTCCTGCCCATCCAGCACCAGGACTGGCT | |
| | GAAGGGGAAGGAGTTCAAGTGCAAGGTCAACAACAAAGACCTCG | |
| | TTTCCCCCATCACGAGGACCATCTCCAAGGCTACAGGGCCGAGC | |
| | CGGGTGCCGCAGGTGTACACCCTGCCCCCAGCCTGGGAAGAGC | |
| | TGTCCAAGAGCAAAGTCAGCATAACCTGCCTGGTCACTGGCTTCT | |
| | ACCCACCTGACATCGATGTCGAGTGGCAGAGCAACGGACAACAA | |
| | GAGCCAGAGGGCAATTACCGCACCACCCCGCCCCAGCAGGACG | |
| | TGGATGGGACCTACTTCCTGTACAGCAAGCTCGCGGTGGACAAG | |
| | GTCAGGTGGCAGCGTGGAGACCTATTCCAGTGTGCGGTGATGCA | |
| | CGAGGCTCTGCACAACCACTACACCCAGAAGTCCATCTCCAAGAG | |
| | TCAGGGTAAA | |

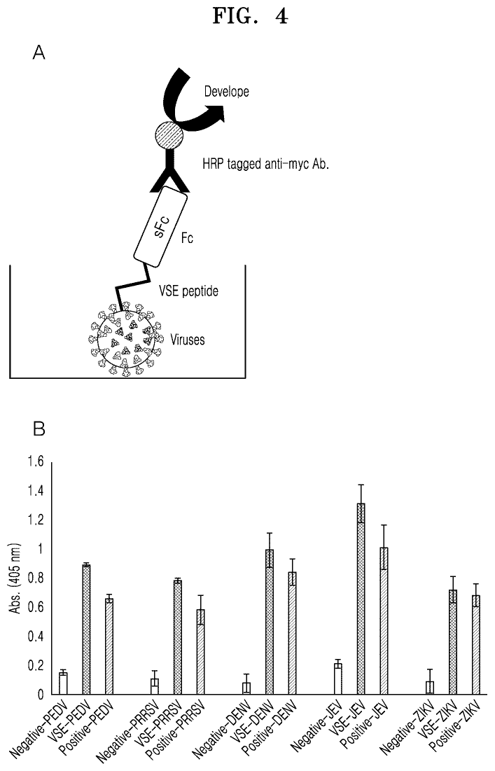

Afterwards, porcine epidemic diarrhea virus (PEDV), porcine reproductive and respiratory syndrome virus (PRRSV), dengue virus (DENV), Japanese encephalitis virus (JEV), or Zika virus (ZIKV) were each coated on the surface of the immunoplate, followed by the addition of VSE-hFc or VSE-sFc to induce an adhesion/binding reaction. Afterwards, Horseradish peroxidase (HRP)-labeled anti-IgG antibody was added to induce the reaction, and the level of HRP was quantitatively detected to assess the adhesion activity to the virus surface (A of FIG. 4). Meanwhile, the negative control group was set as the group using scrambled peptide and the positive control group was set as the group using mouse serum immunized with each virus.

As a result, as shown in B of FIG. 4, the negative control group had a very low detection level of labeled HRP, while the group using the VSE peptide (VSE-hFc, VSE-sFc) according to one aspect detected a level similar to that of the positive control group. These experimental results indicate that the VSE peptide according to one aspect may contribute to improving the efficacy of a vaccine formulation including a viral antigen by attaching or binding an immune-enhancing substance to the surface of the virus antigen.

### Example 4: Confirmation of Immune Response Enhancement Effect Through Virus Surface Engineering

In this embodiment, a recombinant antigen was prepared by attaching/binding VSE-sFc to the surface of the virus antigen, and then the immune response enhancing effect of the recombinant antigen was confirmed using a mouse model.

### 4-1. PEDV-VSE-sFc

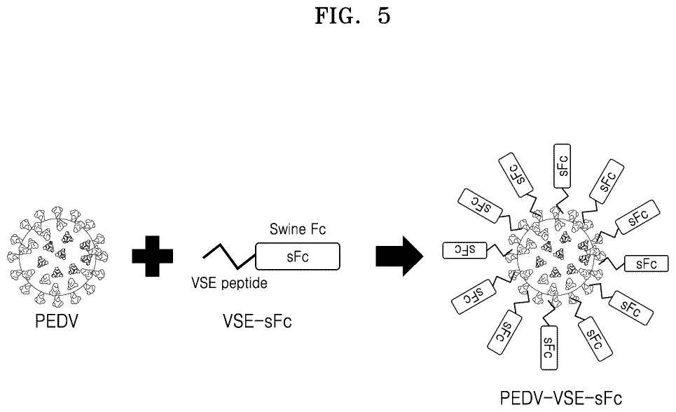

As shown in FIG. 5, porcine epidemic diarrhea virus (PEDV) and VSE-sFc of Example 3 were mixed, and an adhesion/binding reaction was induced between the two at room temperature for 2 hours to prepare a recombinant antigen (PEDV-VSE-sFc).

Specifically, 4-week-old Balb/C mice were immunized with PEDV-VSE-sFc by intraperitoneal administration three times at 2-week intervals, and then the level of specific IgG against PEDV present in the mouse serum was confirmed. In addition, a plaque reduction neutralization test was performed on the serum of the immunized mouse to evaluate the level of neutralizing antibodies against PEDV antigen. Meanwhile, the group administered with PBS was set as the negative control group, and the group administered only with PEDV was set as the comparison group.

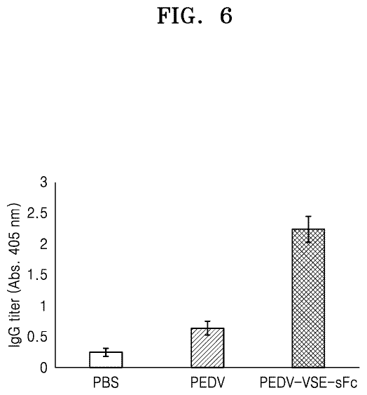

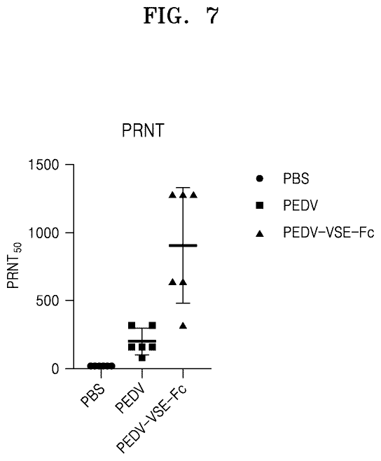

As a result, as shown in FIG. 6 and FIG. 7, high levels of IgG and neutralizing antibodies were confirmed in the mouse serum of the group administered with PEDV-VSE-sFc according to one aspect. In particular, the level of neutralizing antibodies in the serum of mouse immunized with PEDV-VSE-sFc was increased by about 4.5 times compared to the comparison group.

### 4-2. DENV-VSE-hFc

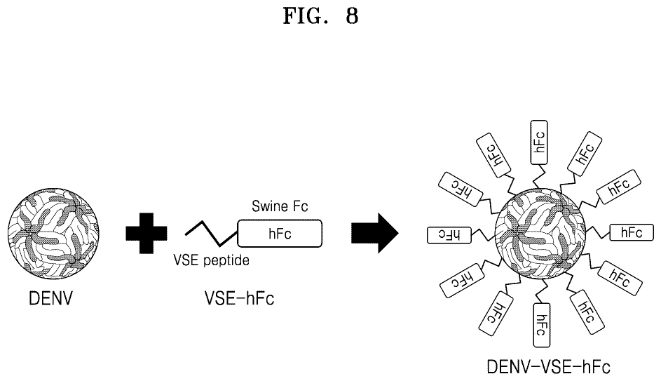

As shown in FIG. 8, a recombinant antigen (DENV-VSE-hFc) was prepared by mixing dengue virus (DENV) with VSE-hFc of Example 3 and inducing an adhesion/binding reaction between the two for 2 hours at room temperature.

Specifically, 4-week-old Balb/C mice were immunized with DENV-VSE-hFc by intraperitoneal administration three times at 2-week intervals, and then the level of specific IgG against DENV present in the mouse serum was confirmed. In addition, a plaque reduction neutralization test was performed on the serum of the immunized mouse to evaluate the level of neutralizing antibodies against DENV antigens. Meanwhile, the group administered with PBS was set as the negative control group, and the group administered only with DENV was set as the comparison group.

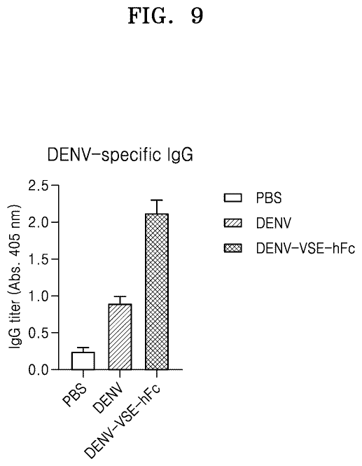

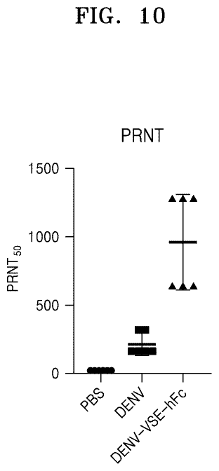

As a result, as shown in FIG. 9 and FIG. 10, high levels of IgG and neutralizing antibodies were confirmed in the mouse serum of the group administered with DENV-VSE-hFc according to one aspect. In particular, the level of neutralizing antibodies in the serum of mouse immunized with DENV-VSE-hFc was increased by about 4.5 times compared to the comparison group.

Summarizing these experimental results, it was found that when an immune-enhancing substance was attached to the surface of a virus using the VSE peptide according to one aspect, the effect of inducing an immune response to the viral antigen was significantly improved. Accordingly, the above recombinant antigen shows improved effectiveness as an active ingredient in vaccine preparations.

### Example 5: Confirmation of Effect of Enhancing Immune Response to Virus-Derived Antigen

In this embodiment, a recombinant antigen was prepared by attaching/binding VSE-sFc to a virus-derived antigen, and then the immune response enhancing effect of the recombinant antigen was confirmed.

### 5-1. PEDV-Derived Spike Protein

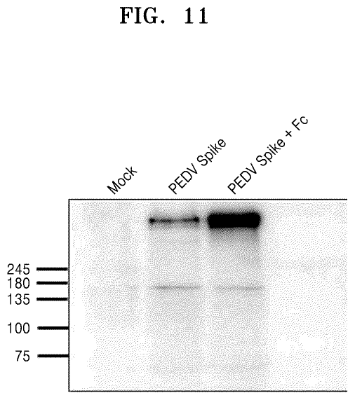

A recombinant antigen including a spike protein S1-derived protein of PEDV as an antigen that may induce a vaccine response to PEDV and an Fc-derived protein of IgG as an immune-enhancing substance to promote antibody formation was produced in the same manner as in Example 4 above using a VSE peptide. In addition, the recombinant antigen was expressed in CHO cells to obtain PEDV-Fc, a recombinant antigen including an immune-enhancing substance, as shown in FIG. 11. The amino acid sequence of the VSE peptide-Fc-derived protein used for the above recombinant antigen (PEDV-Fc) is shown in Table 4 below.

TABLE 4

| NAME | AMINO ACID SEQUENCE (5′→3′) | SEQ ID NO: |
|---|---|---|
| PEDV-Fc | TQEVYDTHDCATNGTIRPFKVLSMRSLIYFWLLLPVLPTLSLPQDVTRC | 7 |
| | QSTTNFRRFFSKFNVQAPAVVVLGGYLPSMNSSSWYCGTGIETASGV | |
| | HGIFLSYIDSSQGFEIGISQEPFDPSGYQLYLHKATNGNTNAIARLRISQF | |
| | PDNKTLGPTVNDVTTGRNCLFNKAIPAYMRDGKDIVVGITWDNDRVTV | |
| | FADKIYHFYLKNDWSRVATRCYNRRSCAMQYVYTPTYYMLNVTSAGE | |
| | DGIYYEPCTANCTGYAANVFATDSNGHIPESFSFNNWFLLSNDSTLLHG | |
| | KVVSNQPLLVNCLLAIPKIYGLGQFFSFNHTMDGVCNGAALDRAPEALR | |
| | FNINDTSVILAEGSIVLHTALGTNLSFVCSNSSDPHLATFAIPLGATEVPY | |
| | YCFLIVDTYNSTVYKFLAVLPPTVREIVITKYGDVYVNGFGYLHLGLLDA | |
| | VTINFTGHGTDDDVSGFWTIDSTNFVDALIEVQGTSIQRILYCDDPVSQL | |
| | KCSQVAFDLDDGFYPISSRNLLSHEQPISFVTLPSFNDHSFVNITVSASF | |
| | GDHSGANLVASDTTINGFSSFCVDTRQFTIRLFYNVTSSYGYVSKSQYS | |
| | NCPFTLQSVNDYLSFSKFCVSTSLLASACTIDLFGYPHFGSGVKFTSLY | |
| | FQFTEGELITGTPKPLEGVTDVSFMTLDVCTKYTIYGFKGEGIITLTNSSF | |
| | LAGVYYTSDSGQLLÅFKNVTSGAVYSVTPCSFSEQAAYVDDDIVGVISS | |
| | LSNSTFNNTRELPGFFYHSNDGSNCTEPVLVYSNIGVCKSGSIGYVPSQ | |
| | SGQVKIAPTVTGNISIPTNFSMSIRTEYLQLYNTPVSVDCATYVQNGNSR | |
| | CKQLLTQYTAACKTIESALQLSARLESVEVNSMLTTSEQALQLÅTISSFN | |
| | GDGYNFTNVLGVSVYDPASGRVVHKRSFIEDLLFNKVVINGLGTVDED | |
| | YKRCSNGRSVADLVCAQYYSGVMVLPGVVDAEKLHMYSASLIGGMVL | |
| | GGFTAAAALPFSYAVQARLNYLALQTDVLQRNQQLLAESFNSAIGNITS | |
| | AFESVKEAISQTSQGLNTVARALTKVQEVVNSQGAALTQLTVQLQHNF | |
| | QAISSSIDDIYSRLDILSADVQVDRLITGRLSALNAFVAQTLTKYTEVQAS | |
| | RKLAQQKVNECVKSQSQRYGFCGGDGEHIFSLVQAAPQGLLFLHTVLV | |
| | PGDFVNVIAIAGLCVNGDIALTLREPGLVLFTHELQTHTATEYFVSSRRM | |
| | FELRKPTVSDFVQIESCVVTYVNLTSDQLPDVIPDYIDVNKTLDEILASLP | |
| | NRTGPSLPLDVFNATYLNLTGEIADLEQRSESLQNTTEELRTLIYNINNTL | |
| | VDLEWLNRVETYIKWPWWIWLIIFIVLIFVVSLLVFCCISTGCCGCCGCC | |
| | GACFSGCCRGPRLQPYEAFEKVHVQDIEPPTPICPEICSCPAAEVLGAP | |
| | SVFLFPPKPKDILMISRTPKVTCVVVDVSQEEAEVQFSWYVDGVQLYTA | |
| | QTRPMEEQFNSTYRVVSVLPIQHQDWLKGKEFKCKVNNKDLLSPITRTI | |
| | SKATGPSRVPQVYTLPPAWEELSKSKVSITCLVTGFYPPDIDVEWQSN | |
| | GQQEPEGNYRTTPPQQDVDGTYFLYSKLAVDKVRWQRGDLFQCAVM | |
| | HEALHNHYTQKSISKTQGK | |

In addition, the following experiment was performed to confirm the immunogenicity of the immune-enhanced PEDV virus vaccine. The prepared vaccine (PEDV-Fc) was intramuscularly inoculated twice at 2-week intervals in experimental animals (administration dose: 100 ul). Two weeks after the second vaccination, serum and colostrum were collected to measure IgG titer, and ELISA and neutralizing ability tests were performed to examine antibody titers in serum and colostrum. Meanwhile, a group to which PBS was added was set as a control group, and a group to which only PEDV was administered was set to be a comparison group.

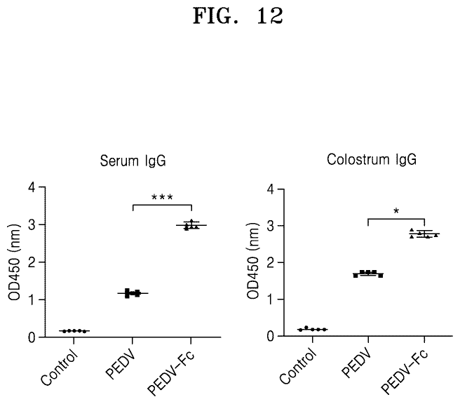

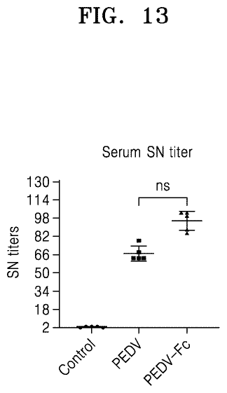

As a result, as shown in FIG. 12 and FIG. 13, the recombinant antigen according to an embodiment showed a higher level of IgG titer in the serum and colostrum of sows compared to the comparison group, and the neutralizing antibody titer in the serum also showed the same trend as above.

### 5-2. PRRSV-Derived GP5 Protein

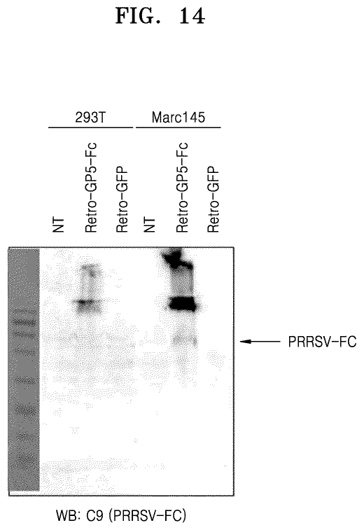

A recombinant antigen including a GP5 protein of PRRSV as an antigen that may induce a vaccine response to PRRSV and an Fc-derived protein of IgG as an immune-enhancing substance to promote antibody formation was produced in the same manner as in Example 4 above using a VSE peptide. In addition, the recombinant antigen was expressed in CHO cells and Marc 145 cells to obtain PRRSV-Fc, a recombinant antigen including an immune-enhancing substance, as shown in FIG. 14. The amino acid sequence of the VSE peptide-Fc-derived protein used for the above recombinant antigen (PRRSV-Fc) is shown in Table 4 below.

TABLE 5

| NAME | AMINO ACID SEQUENCE (5′→3′) | SEQ ID NO: |
|---|---|---|
| PRRSV-Fc | TQEVYDTHDCATNGTIRPFKVLSMLEKCLTAGCYSQLLS | 8 |
| | LWCIVPFCFAVLVNAAPKTAPSVYPLAPCGRDVSGPNVA | |
| | LGCLASSYFPEPVTVTWNSGALTSGVHTFPSVLQPSGLY | |
| | SLSSMVTVPASSLSSKSYTCNVNHPATTTKVDKRVGIHQ | |
| | PQTCPCPGCEVAGPSVFIFPPKPKDTLMISQTPEVTCVV | |
| | VDVSKEHAEVQFSWYVDGVEVHTAETRPKEEQFNSTYR | |
| | VVSVLPIQHQDWLKGKEFKCKVNNVDLPAPITRTISKAIG | |
| | QSREPQVYTLPPPAEELSRSKVTLTCLVIGFYPPDIHVEW | |
| | KSNGQPEPENTYRTTPPQQDVDGTFFLYSKLAVDKARW | |
| | DHGDKFECAVMHEALHNHYTQKSISKTQGKYVLSSIYAV | |
| | CALAALTCFVIRFAKNCMSWRYACTRYTNFLDTKGRLY | |
| | RWRSPVIIEKRGKVEVEGHLIDLKRVVLDGSVATPITRVS | |
| | AEQWGRP | |

In addition, the following experiment was performed to confirm the immunogenicity of the immune-enhanced PRRS virus vaccine. The prepared vaccine (PRRSV-Fc) was intramuscularly inoculated twice at 2-week intervals in experimental animals (administration dose: 100 ul). Two weeks after the second vaccination, serum and colostrum were collected to measure IgG titer, and ELISA and neutralizing ability tests were performed to examine antibody titers in serum and colostrum. Meanwhile, a group to which PBS was added was set as a control group, and a group to which only PRRSV was administered was set to be a comparison group.

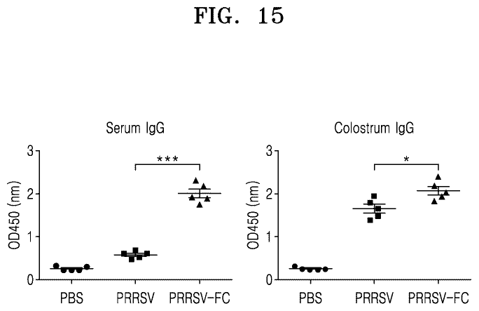

As a result, as shown in FIG. 15, the recombinant antigen according to an embodiment showed a higher level of IgG titer in both the sow's serum and colostrum compared to the comparison group.

The above experimental results confirm that the vaccine composition according to an embodiment has increased immunogenicity, which may maximize the efficacy of the vaccine.

The foregoing description of the disclosure is for illustrative purposes only, and one that has ordinary skill in the art to which the disclosure belongs will understand that it may be readily adapted to other specific forms without altering the technical ideas or essential features of the disclosure. Therefore, the embodiments described above should be understood in all respects as illustrative and not restrictive.

1. A linker peptide consisting of an amino acid sequence of SEQ ID NO: 1.

2. A fusion protein comprising: a linker peptide consisting of an amino acid sequence of SEQ ID NO: 1; and
an immune-enhancing substance connected to a C-terminus of the linker peptide.

3. The fusion protein of claim 2, wherein the immune-enhancing substance is any one or more selected from an Fc region of an antibody, flagellin, or interleukin-2 (IL-2).

4. The fusion protein of claim 2, wherein the immune-enhancing substance is an Fc region of an antibody.

5. A polynucleotide encoding the fusion protein of claim 2.

6. A recombinant vector comprising the polynucleotide of claim 5.

7. A host cell transformed with the recombinant vector of claim 6.

8. A vaccine composition comprising: an infectious virus-derived antigen; and
a fusion protein including a linker peptide consisting of an amino acid sequence of SEQ ID NO: 1 and an immune-enhancing substance connected to a C-terminus of the linker peptide.

9. The vaccine composition of claim 9, wherein an N-terminus of the linker peptide is connected to an infectious virus-derived antigen.

10. The vaccine composition of claim 9, wherein the infectious virus-derived antigen is an antigen derived from Porcine epidemic diarrhea virus, Porcine reproductive and respiratory syndrome virus, Dengue virus, Japanese encephalitis virus, Zika virus, Ebola virus, Rotavirus, West Nile virus, Yellow fever virus, Adenovirus, BK virus, Smallpox virus, Severe fever with thrombocytopenia syndrome virus, Herpes simplex virus, Epstein-Barr virus, Hepatitis A virus, Hepatitis B virus, Hepatitis C virus, Hepatitis D virus, Hepatitis E virus, Hantan virus, or Cytomegalovirus.

11. The vaccine composition of claim 9, wherein the vaccine composition is a live attenuated vaccine, an inactivated vaccine, a subunit vaccine, or a virus-like particle vaccine.


SEQUENCE LISTING (8 sequences)

SEQ ID NO: 1 - protein, 23 aa, Artificial
  VSE peptide
TQEVYDTHDCATNGTIRPFKVLS

SEQ ID NO: 2 - DNA, 69 bp, Artificial
  VSE polynucleotide
acccaagaggtgtacgacacccacgactgcgccaccaacggcaccatcagacctttcaaggtgctgagc

SEQ ID NO: 3 - protein, 250 aa, Artificial
  VSE-human Fc peptide
TQEVYDTHDCATNGTIRPFKVLSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 750 bp, Artificial
  VSE-human Fc polynucleotide
acccaagaggtgtacgacacccacgactgcgccaccaacggcaccatcagacctttcaaggtgctgagcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 5 - protein, 356 aa, Artificial
  VSE-swine Fc peptide
TQEVYDTHDCATNGTIRPFKVLSAYNTAPSVYPLAPCGRDVSDHNVALGCLVSSYFPEPVTVTWNSGALSRVVHTFPSVLQPSGLYSLSSMVIVAASSLSTLSYTCNVYHPATNTKVDKRVDIEPPTPICPEICSCPAAEVLGAPSVFLFPPKPKDILMISRTPKVTCVVVDVSQEEAEVQFSWYVDGVQLYTAQTRPMEEQFNSTYRVVSVLPIQHQDWLKGKEFKCKVNNKDLLSPITRTISKATGPSRVPQVYTLPPAWEELSKSKVSITCLVTGFYPPDIDVEWQSNGQQEPEGNYRTTPPQQDVDGTYFLYSKLAVDKVRWQRGDLFQCAVMHEALHNHYTQKSISKTQGK

SEQ ID NO: 6 - DNA, 1068 bp, Artificial
  VSE-swine Fc polynucleotide
acccaagaggtgtacgacacccacgactgcgccaccaacggcaccatcagacctttcaaggtgctgagcgcctacaacacagctccatcggtctaccctctggccccctgtggcagggacgtgtctgatcataacgtggccttgggctgccttgtctcaagctacttccccgagccagtgaccgtgacctggaactcgggtgccctgtccagagtcgtgcataccttcccatccgtcctgcagccgtcagggctctactccctcagcagcatggtgatcgtggcggccagcagcctgtccaccctgagctacacgtgcaacgtctaccacccggccaccaacaccaaggtggacaagcgtgttgacatcgaaccccccacacccatctgtcccgaaatttgctcatgcccagctgcagaggtcctgggagcaccgtcggtcttcctcttccctccaaaacccaaggacatcctcatgatctcccggacacccaaggtcacgtgcgtggtggtggacgtgagccaggaggaggctgaagtccagttctcctggtacgtggacggcgtacagttgtacacggcccagacgaggccaatggaggagcagttcaacagcacctaccgcgtggtcagcgtcctgcccatccagcaccaggactggctgaaggggaaggagttcaagtgcaaggtcaacaacaaagacctcctttcccccatcacgaggaccatctccaaggctacagggccgagccgggtgccgcaggtgtacaccctgcccccagcctgggaagagctgtccaagagcaaagtcagcataacctgcctggtcactggcttctacccacctgacatcgatgtcgagtggcagagcaacggacaacaagagccagagggcaattaccgcaccaccccgccccagcaggacgtggatgggacctacttcctgtacagcaagctcgcggtggacaaggtcaggtggcagcgtggagacctattccagtgtgcggtgatgcacgaggctctgcacaaccactacacccagaagtccatctccaagactcagggtaaa

SEQ ID NO: 7 - protein, 1641 aa, Artificial
  PEDV-Fc
TQEVYDTHDCATNGTIRPFKVLSMRSLIYFWLLLPVLPTLSLPQDVTRCQSTTNFRRFFSKFNVQAPAVVVLGGYLPSMNSSSWYCGTGIETASGVHGIFLSYIDSSQGFEIGISQEPFDPSGYQLYLHKATNGNTNAIARLRISQFPDNKTLGPTVNDVTTGRNCLFNKAIPAYMRDGKDIVVGITWDNDRVTVFADKIYHFYLKNDWSRVATRCYNRRSCAMQYVYTPTYYMLNVTSAGEDGIYYEPCTANCTGYAANVFATDSNGHIPESFSFNNWFLLSNDSTLLHGKVVSNQPLLVNCLLAIPKIYGLGQFFSFNHTMDGVCNGAALDRAPEALRFNINDTSVILAEGSIVLHTALGTNLSFVCSNSSDPHLATFAIPLGATEVPYYCFLIVDTYNSTVYKFLAVLPPTVREIVITKYGDVYVNGFGYLHLGLLDAVTINFTGHGTDDDVSGFWTIDSTNFVDALIEVQGTSIQRILYCDDPVSQLKCSQVAFDLDDGFYPISSRNLLSHEQPISFVTLPSFNDHSFVNITVSASFGDHSGANLVASDTTINGFSSFCVDTRQFTIRLFYNVTSSYGYVSKSQYSNCPFTLQSVNDYLSFSKFCVSTSLLASACTIDLFGYPHFGSGVKFTSLYFQFTEGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSNSTFNNTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTTSEQALQLATISSFNGDGYNFTNVLGVSVYDPASGRVVHKRSFIEDLLFNKVVTNGLGTVDEDYKRCSNGRSVADLVCAQYYSGVMVLPGVVDAEKLHMYSASLIGGMVLGGFTAAAALPFSYAVQARLNYLALQTDVLQRNQQLLAESFNSAIGNITSAFESVKEAISQTSQGLNTVARALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPGDFVNVIAIAGLCVNGDIALTLREPGLVLFTHELQTHTATEYFVSSRRMFELRKPTVSDFVQIESCVVTYVNLTSDQLPDVIPDYIDVNKTLDEILASLPNRTGPSLPLDVFNATYLNLTGEIADLEQRSESLQNTTEELRTLIYNINNTLVDLEWLNRVETYIKWPWWIWLIIFIVLIFVVSLLVFCCISTGCCGCCGCCGACFSGCCRGPRLQPYEAFEKVHVQDIEPPTPICPEICSCPAAEVLGAPSVFLFPPKPKDILMISRTPKVTCVVVDVSQEEAEVQFSWYVDGVQLYTAQTRPMEEQFNSTYRVVSVLPIQHQDWLKGKEFKCKVNNKDLLSPITRTISKATGPSRVPQVYTLPPAWEELSKSKVSITCLVTGFYPPDIDVEWQSNGQQEPEGNYRTTPPQQDVDGTYFLYSKLAVDKVRWQRGDLFQCAVMHEALHNHYTQKSISKTQGK

SEQ ID NO: 8 - protein, 476 aa, Artificial
  PRRSV-Fc
TQEVYDTHDCATNGTIRPFKVLSMLEKCLTAGCYSQLLSLWCIVPFCFAVLVNAAPKTAPSVYPLAPCGRDVSGPNVALGCLASSYFPEPVTVTWNSGALTSGVHTFPSVLQPSGLYSLSSMVTVPASSLSSKSYTCNVNHPATTTKVDKRVGIHQPQTCPCPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTLTCLVIGFYPPDIHVEWKSNGQPEPENTYRTTPPQQDVDGTFFLYSKLAVDKARWDHGDKFECAVMHEALHNHYTQKSISKTQGKYVLSSIYAVCALAALTCFVIRFAKNCMSWRYACTRYTNFLLDTKGRLYRWRSPVIIEKRGKVEVEGHLIDLKRVVLDGSVATPITRVSAEQWGRP